(19) United States
(12) Patent Application Publication
(10) Pub. No.: US 2025/0014777 A1
(43) Pub. Date: Jan. 9, 2025

(54) ELECTRODE AND ELECTRODE KIT

(71) Applicant: BEX CO., LTD., Tokyo (JP)

(72) Inventors: Yusuke SAITO, Tokyo (JP); So MAKABE, Tokyo (JP); Yasuhiro MORIIZUMI, Tokyo (JP)

(21) Appl. No.: 18/579,839

(22) PCT Filed: Jun. 13, 2022
(86) PCT No.: PCT/JP22/23577

§ 371 Date: Sep. 24, 2024

(30) Foreign Application Priority Data
Jul. 19, 2021 (JP) 2021-118393

(51) Int. Cl. H01B 5/00; C12M 1/42

(52) U.S. Cl. CPC H01B 5/002; C12M 35/02

(57) ABSTRACT

An object is to provide an electrode that is easily arranged at a predetermined position when electroporation is performed. The object is achieved by an electrode used for performing electroporation, the electrode includes: at least two or more conductors; and a member configured to hold each conductor and expose at least one end of each of the conductors to outside, the member has a first face where the one end of each of the conductors is exposed, and at least one or more protruding parts provided to the first face, and the length by which the one or more protruding parts protrude in an axis direction from the first face is longer than the length by which the one end of each of the conductors is exposed from the first face.

### TECHNICAL FIELD

The disclosure in the present application relates to an electrode and an electrode kit.

### BACKGROUND ART

Methods to introduce a biological substance such as a nucleic acid molecule (such as DNA and RNA) or a protein, a compound serving as an active ingredient of a drug, or the like into target cells as a foreign substance have been widely developed. In particular, gene transfer technologies to introduce a nucleic acid molecule into cells are basic technologies in genetic engineering. Thus, the gene transfer technologies are needed in wide range of fields such as genetically modified crops, gene therapy, genome analysis, genome editing technology, and the like.

Schemes of gene transfer technologies can be classified into biological schemes, chemical schemes, and physical schemes. Among other things, the physical schemes have an advantage that it is less required to take toxicity to cells into consideration than the biological schemes and the chemical schemes and there is no limitation of applicable cells. In particular, an electroporation method is a scheme that is the most versatile and widespread in the physical schemes.

In general electroporation, a cell suspension is put into a cuvette in which two plate electrodes are arranged, and electric pulses are applied thereto. Thus, when introducing molecules into adherent cells, processes of detaching cells from a substrate or the like to prepare the cell suspension, applying an electric field to the cells, and then re-seeding the cells to the substrate or the like to cultivate the cells are performed. However, enzyme treatment with trypsin or the like for detaching cells may cause damage to a protein and a cytoskeleton present on the cell membrane or the cell membrane surface. Accordingly, to avoid damage to cells due to detachment, an electrode with legs to perform electroporation without detaching adherent cells from a substrate or the like has been developed as disclosed in Patent Literature 1.

### CITATION LIST

### Patent Literature

Patent Literature 1: Japanese Patent No. 4713671

### SUMMARY OF INVENTION

### Technical Problem

In the electroporation on adherent cells disclosed in Patent Literature 1, electrodes with legs are arranged on cells cultured on a plate, and electric pulses are applied to perform electroporation with the cells being adhered. FIG. 8 illustrates a conventional electrode with legs. FIG. 8A illustrates the whole electrode with legs. FIG. 8B illustrates a schematic diagram of a side face of the tip of the electrode with legs when viewed from front. FIG. 8C illustrates a schematic diagram of the bottom face of the electrode with legs when viewed from front. In the electrode with legs, a conductor 81 is coated with an insulator 82 made of epoxy or the like, and the conductor 81 is exposed at the tip. Further, the electrode with legs has legs 83 at the tip. Because the electrode with legs has the legs 83, cells adhered to a substrate or the like and the conductor 81 can be maintained at a constant distance, and electroporation can be performed without the conductor 81 being in contact with the cells. However, while the electrode with legs does not cause contact between the conductor 81 and the cells, the portion of the legs 83 in contact with the substrate or the like is small. Thus, when the electrode with legs is arranged in contact with the substrate or the like, the electrode with legs is likely to be unsettled, and this makes it difficult to maintain the electrode with legs at a predetermined position. Therefore, when an electrode with legs is used, since it is difficult to maintain the electrode with legs (conductor 81) at a predetermined position, the distance between the conductor 81 and the substrate to which cells are adhered is unstable for each experiment, and this causes a problem of difficulty in performing electroporation in the same condition.

Accordingly, an object of the disclosure in the present application is to provide an electrode and an electrode kit that can be easily arranged at a predetermined position. Other optional, additional advantageous effects of the disclosure in the present application will be apparent in embodiments of the present invention.

### Solution to Problem

(1) An electrode used for performing electroporation, the electrode comprising:
at least two or more conductors; and
a member configured to hold each conductor and expose at least one end of each of the conductors to outside,
wherein the member has
a first face where the one end of each of the conductors is exposed, and
at least one or more protruding parts provided to the first face, and
wherein the length by which the one or more protruding parts protrude in an axis direction from the first face is longer than the length by which the one end of each of the conductors is exposed from the first face.

(2) The electrode according to (1) above, wherein the one or more protruding parts comprise two or more protruding parts, and the two or more protruding parts are spaced apart from each other.

(3) The electrode according to (2) above, wherein when, in an outer circumferential portion of the first face, a portion interposed between virtual lines extending from the conductors facing each other is defined as an outer circumferential virtual region,
a clearance, which is provided by the protruding parts being spaced apart from each other, and the outer circumferential virtual region at least partially overlap each other.

(4) The electrode according to (1) above, wherein a slope part is provided to the first face.

(5) The electrode according to (2) above, wherein a slope part is provided to the first face.

(6) The electrode according to (3) above, wherein a slope part is provided to the first face.

(7) The electrode according to any one of (1) to (6) above, wherein the one or more protruding parts are arranged on the outer circumferential side from each of the conductors exposed in the first face.

(8) The electrode according to any one of (1) to (6) above,
wherein the member has a through hole and/or a recess in the first face,
wherein the through hole penetrates between the first face and a face other than the first face, and
wherein the recess is recessed in an axis direction of the member from the first face and penetrates through in a side face direction of the member.

(9) The electrode according to (7) above,
wherein the member has a through hole and/or a recess in the first face,
wherein the through hole penetrates between the first face and a face other than the first face, and
wherein the recess is recessed in an axis direction of the member from the first face and penetrates through in a side face direction of the member.

(10) An electrode kit comprising:
the electrode according to any one of (1) to (6) above; and
a holder connected to a power supply,
wherein the other end of each of the conductors is electrically connected to the holder, and power is supplied to each of the conductors from the power supply.

(11) An electrode kit comprising:
the electrode according to (7) above; and
a holder connected to a power supply,
wherein the other end of each of the conductors is electrically connected to the holder, and power is supplied to each of the conductors from the power supply.

(12) An electrode kit comprising:
the electrode according to (8) above; and
a holder connected to a power supply,
wherein the other end of each of the conductors is electrically connected to the holder, and power is supplied to each of the conductors from the power supply.

(13) An electrode kit comprising:
the electrode according to (9) above; and
a holder connected to a power supply,
wherein the other end of each of the conductors is electrically connected to the holder, and power is supplied to each of the conductors from the power supply.

### Advantageous Effect

The electrode disclosed in the present application can be easily arranged at a predetermined position when electroporation is performed on cells.

### BRIEF DESCRIPTION OF DRAWINGS

FIG. 1A is a diagram schematically illustrating an example of the external appearance of an electrode 1A.

FIG. 1B is a diagram of a first face 31 of the electrode 1A when viewed from front. FIG. 1C is a sectional view taken along the arrow X-X′ of FIG. 1B.

FIG. 2A is a diagram of the first face 31 of an electrode 1B when viewed from front. FIG. 2B is a sectional view taken along the arrow X-X′ of FIG. 2A.

FIG. 3A is a diagram of the first face 31 of an electrode 1C when viewed from front. FIG. 3B is a sectional view taken along the arrow Y-Y′ of FIG. 3A.

FIG. 4 represents diagrams illustrating protruding parts 34 provided to the first face 31 of a member 3 as examples.

FIG. 5 represents schematic diagrams of electrodes 1D to 1F in which the member 3 has recesses 37 or a through hole 38. FIG. 5A is a diagram of the first face 31 of the electrode 1D when viewed from front. FIG. 5B is a sectional view taken along the arrow X-X′ of FIG. 5A. FIG. 5C is a diagram of the first face 31 of the electrode 1E when viewed from front. FIG. 5D is a sectional view taken along the arrow X-X′ of FIG. 5C.

FIG. 5E is a diagram of the first face 31 of the electrode 1F when viewed from front. FIG. 5F is a sectional view taken along the arrow X-X′ of FIG. 5E.

FIG. 6 represents schematic diagrams of an electrode 1 used in Example 1. FIG. 6A is a diagram of the first face 31 of the electrode 1 when viewed from front. FIG. 6B is a sectional view taken along the arrow X-X′ of FIG. 6A.

FIG. 7 represents photographs substitute for drawings, which are fluorescence micrographs of adherent cells after electroporation.

FIG. 8 represents diagrams illustrating the conventional electrode with legs. FIG. 8A is a photograph substitute for a drawing, which is a photograph illustrating the conventional electrode with legs. FIG. 8B is a schematic diagram of the side face of the conventional electrode with legs when viewed from front. FIG. 8C is a schematic diagram of the bottom face of the conventional electrode with legs when viewed from front.

### DETAILED DESCRIPTION OF EMBODIMENTS

Embodiments of an electrode and an electrode kit will be described below in detail with reference to the drawings. Note that, in the present specification, members having similar functions are labeled with the same or similar references. Further, for the members labeled with the same or similar references, duplicated description may be omitted.

Further, the position, the size, the range, or the like of each configuration illustrated in the drawings do not always represent the actual position, the actual size, the actual range, or the like for easier understanding. Thus, the disclosure in the present application is not necessarily limited to the position, the size, the range, like disclosed in the drawings.

### First Embodiment of Electrode

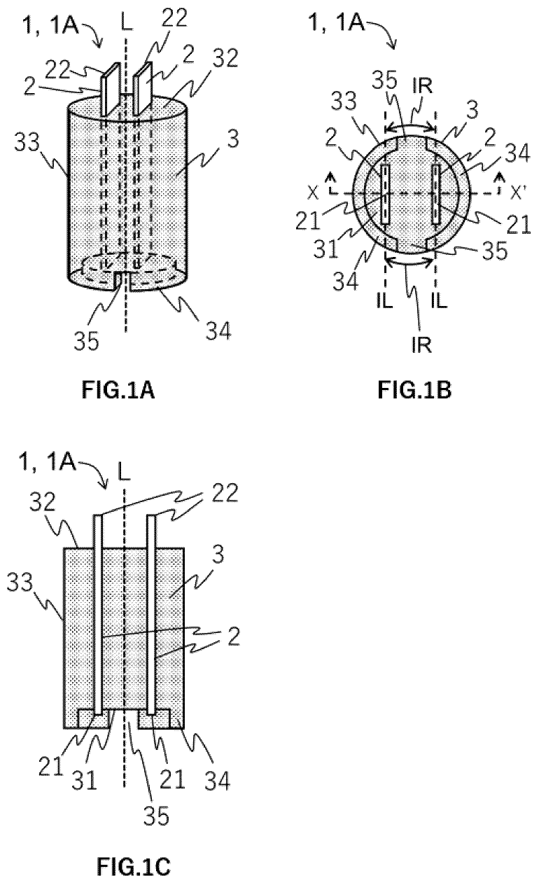

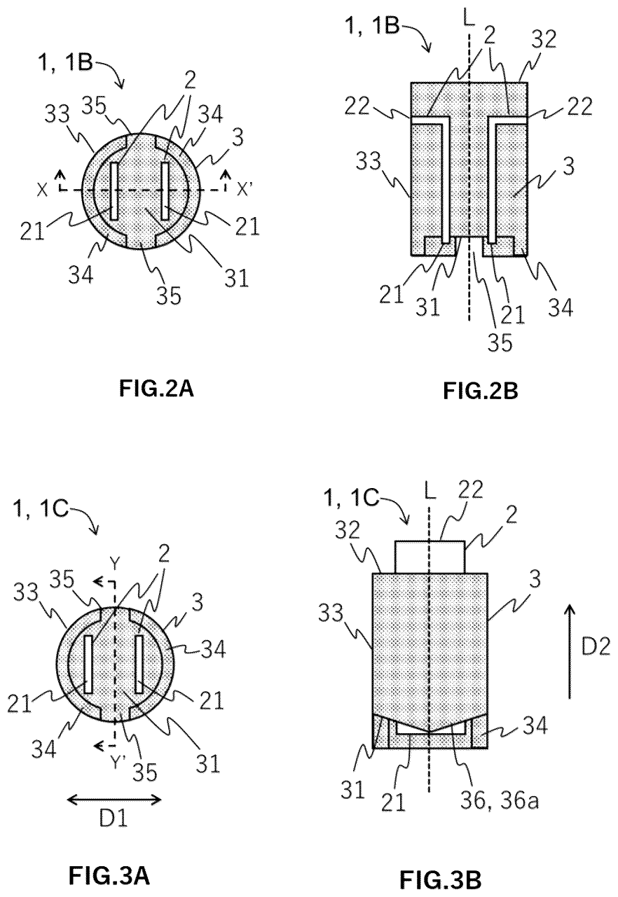

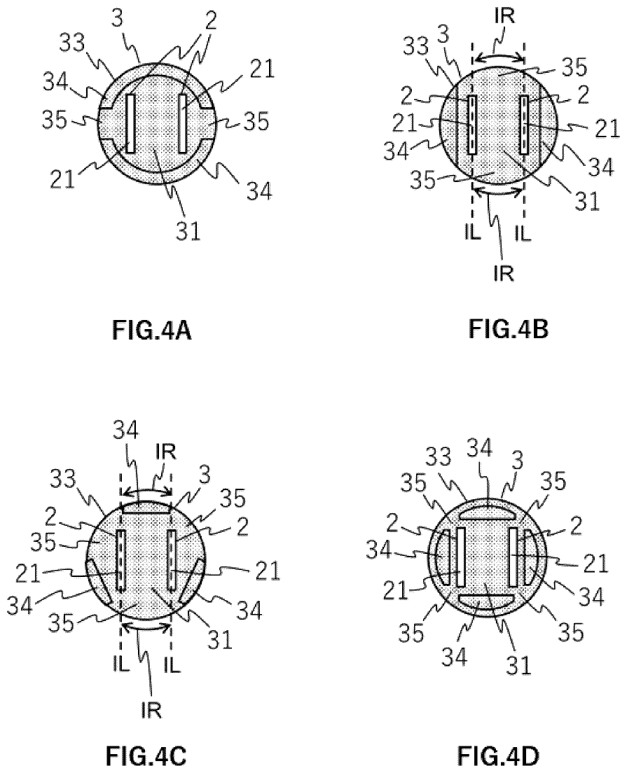

Electrodes 1A to 1C according to a first embodiment will be described with reference to FIG. 1 to FIG. 4. FIG. 1A is a diagram schematically illustrating an example of the external appearance of the electrode 1A according to the first embodiment. FIG. 1B is a diagram of a first face 31 of the electrode 1A when viewed from front. FIG. 1C is a sectional view taken along the arrow X-X′ of FIG. 1B. FIG. 2A is a diagram of the first face 31 of the electrode 1B when viewed from front. FIG. 2B is a sectional view taken along the arrow X-X′ of FIG. 2A. FIG. 3A is a diagram of the first face 31 of the electrode 1C when viewed from front. FIG. 3B is a sectional view taken along the arrow Y-Y′ of FIG. 3A. FIG. 4 is a diagram illustrating protruding parts 34 provided to the first face 31 of a member 3 as an example.

The electrode 1A according to the first embodiment is used when electroporation is performed on cells. The electrode 1 at least has at least two or more conductors 2 (in the following description, two or more conductors may be simply referred to as “conductors”) and a member 3.

The conductors 2 are held in the member 3 described later. Further, the conductors 2 are electrically connected to an external power supply and used to perform electroporation on cells by using electric pulses supplied from the power supply. The conductor 2 may be any conductor as long as it can conduct electric pulses supplied from the power supply, and the shape or the material of the conductor 2 is not particularly limited. The shape of the conductor 2 may be, for example, a plate shape, a bar shape, or the like. In the example illustrated in FIG. 1, the conductor 2 has a plate shape. Further, the material of the conductor 2 may be, for example, gold, platinum, stainless, titanium, chromium, tungsten, carbon, or the like. Note that these two or more conductors 2 may be of the same shape and material or may be of different shapes or materials as long as they can perform electroporation on cells.

The member 3 holds the conductors 2 and exposes at least one ends of the conductors 2 from the surface of the member 3 so that the conductors 2 can perform electroporation on cells. Further, the other ends of the conductors 2 held by the member 3 may be in any form as long as they can be electrically connected to the external power supply and may or may not be exposed from the surface of the member 3. Note that, in the present specification, the face where one ends of the conductors 2 that perform electroporation on adherent cells of the member 3 (hereafter, which may be referred to as “end(s) 21”) is exposed is defined as the first face 31, and the face opposite to the first face 31 is defined as a second face 32.

In the example illustrated in FIG. 1, the other ends of the conductor 2 held by the member 3 (hereafter, which may be referred to as “end(s) 22”, however, references “21” and “22” may be omitted in some drawings to avoid complicating the drawings) is exposed in the second face 32 of the member 3. Alternatively, although not illustrated, the ends 22 of the conductors 2 may not be exposed in the second face 32. In such a case, to enable electrical connection to the external power supply, the member 3 may have an insertion hole passing through from the second face 32 to the ends of the conductors 2. Furthermore, the conductor 2 held by the member 3 may have an L-shape. FIG. 2 illustrates an electrode 1B that holds the L-shaped conductors 2 in the member 3. Since the member 3 of the electrode 1B holds the L-shaped conductors 2, when the ends 21 of the conductors 2 are exposed in the first face 31, the ends 22 of the conductors 2 are exposed in a side face 33 of the member 3 in the example illustrated in FIG. 2. Therefore, in the electrode 1B, the ends 22 of the conductors 2 exposed in the side face 33 of the member 3 are electrically connected to the external power supply.

Although the member 3 has a cylindrical shape in the example illustrated in FIG. 1, the member 3 may have any shape as long as it can hold the conductors 2, and the shape is not particularly limited. The shape of the member 3 may be any shape other than a cylindrical shape, for example, may be a polygonal prism shape. Further, the material of the member 3 is not particularly limited as long as it is an insulator, for example, may be a resin or the like. The resin may be, for example, silicone, polypropylene, polycarbonate, thermosetting urethane, epoxy, acrylic, or the like.

Further, the conductors 2 held by the member 3 are required to have a positive polarity and a negative polarity when electroporation is performed. Therefore, while the member 3 holds at least two conductors 2, this does not limit that the member 3 holds three or more conductors 2. The number of conductors 2 held by the member 3 can be, for example, two, three, four, or the like. Note that, in the conventional electrode with legs, the three conductors 81 are coated with the insulator 82, respectively, as illustrated in FIG. 8. Thus, when it is understood that the insulator 82 corresponds to a member to hold the conductors 81, the insulator 82 is not a member to hold at least two or more conductors 81. It is therefore apparent that the configuration of the electrode 1 disclosed in the present application differs from the configuration of the electrode with legs of the conventional art.

The length by which the end 21 of each conductor 2 is exposed from the first face 31 (in other words, the length of the conductor 2 protruding from the first face 31) is not particularly as limited long as electroporation can be performed on cells. While electroporation can be performed as long as the first face 31 and the end faces of the ends 21 of the conductors 2 are on the same level, it is preferable that the ends 21 of the conductors 2 protrude from the first face 31. When electroporation is performed, the efficiency thereof varies in accordance with the amount of electroporation buffer (hereafter, which may be referred to as “buffer”), the surface area of the conductor 2 exposed from the first face 31, or the like. Therefore, to achieve desired efficiency of electroporation in accordance with the situation where the electrode 1A according to the first embodiment is used, the surface area of the conductor 2 exposed from the first face 31 can be designed as appropriate.

The member 3 has at least one or more protruding parts 34. Each protruding part 34 is provided to the first face 31 of the member 3 and protrudes in the axis L direction of the member 3. In the present specification, the axis (L) direction of the member 3 means the direction of the center axis of a prism when the member 3 has a prism shape such as a circular prism shape or a polygonal prism shape, for example. Note that, when the shape of the member 3 is not a prism, the direction substantially perpendicular to the first face 31 may be the axis direction. Further, when two or more conductors 2 are held by the member 3 so as to be substantially parallel to each other, it can also be said that the axis direction is a direction parallel to the conductors 2.

Further, the length by which the protruding part 34 protrudes in the axis L direction from the first face 31 is longer than the length by which the end 21 of each conductor 2 is exposed from the first face 31. Thus, when the electrode 1A according to the first embodiment is used to perform electroporation on adherent cells adhered to a substrate or the like, since the top of the protruding part 34 is located on the substrate side from the ends 21 of the conductors 2 exposed in the first face 31, the ends 21 of the conductors 2 are not in contact with the adherent cells. Therefore, this prevents adherent cells from being significantly damaged due to supply of electric pulses. Note that the electrode 1A disclosed in the present application can be suitably used for electroporation on adherent cells in an adhered state and can also be used for electroporation on other cells such as floating cells. In such a case, the length by which the protruding part 34 protrudes in the axis L direction from the first face 31 can be adjusted as appropriate.

Further, in the electrode 1A according to the first embodiment, because the member 3 has the protruding parts 34, the protruding parts 34 come into contact with a substrate or the like and keep the attitude of the electrode 1A when the electrode 1A is installed to the substrate or the like. Therefore, this makes it easier to maintain the conductors 2 exposed in the first face 31 of the member 3 at a predetermined position. In the conventional electrode with legs, it is not possible to provide legs to a part other than the tip of the electrode because of the structure thereof. In contrast, in the electrode 1A, since the member 3 holds the conductors 2, it is possible to provide the protruding parts 34 to the first face 31. In the example illustrated in FIG. 1, the protruding parts 34 are provided along the outer circumference of the first face 31 of the member 3. Because the protruding parts 34 are provided along the outer circumference, when the electrode 1A is installed to a substrate or the like, the contact portion between the protruding parts 34 and the substrate or the like is increased, and this makes it easier to maintain the conductors 2 stably in the place where the electrode 1A is installed.

The number of protruding parts 34, the shape of the protruding part 34, and the place to arrange the protruding parts 34 are not particularly limited as long as the conductors 2 are maintained stably at places where the electrode 1A is installed after installation of the electrode 1A. Since the protruding parts 34 protrude from the first face 31, when the electrode 1A is put into the buffer, air may remain in the space surrounded by the first face 31 and the protruding part 34. If air remains, this may cause a problem with electrical conduction between the conductors 2 facing each other. It is therefore preferable to design the protruding parts 34 so that the air around the first face 31A of the member 3 does not remain when the electrode 1A is put into the buffer. In the example illustrated in FIG. 1, two protruding parts 34 are provided spaced apart from each other to form clearances 35 so that the air around the first face 31 of the member 3 does not remain. Note that, as described later, the protruding part 34 may be provided to the entire outer circumference of the first face 31, and a through hole to allow air remaining in the member 3 to escape may be provided.

Further, a slope part 36 may be provided to the first face 31 to facilitate air to move from the first face 31 to outside. FIG. 3 illustrates an example of the electrode 1C in which the slope part 36 is provided to the first face 31 interposed between the conductors 2 facing each other. The slope part 36 is not particularly limited as long as it has a shape that facilitates air of the portion interposed between the conductors 2 facing each other to move in the outer circumferential direction of the member 3 along the slope part 36 due to floating force when the electrode 1C is put into the buffer. In the example illustrated in FIG. 3, the direction that is substantially orthogonal to the axis L direction and substantially orthogonal to the conductors 2 facing each other is defined as D1, and the direction opposite to the direction in which the protruding part 34 protrudes from the first face 31 is defined as D2. In the example illustrated in FIG. 3B, the slope part 36 is inclined in the D2 direction from substantially the center portion of the first face 31 interposed between the conductors 2 facing each other as any slope face 36a is closer to the outer circumference of the member 3 in the D1 direction. Therefore, even when the electrode 1C is put into the buffer, air moves in the outer circumferential direction of the member 3 along the slope part 36 of the first face 31. Note that it is preferable, but is not limited, that the clearances 35 be formed in the inclination direction of the slope part 36.

Note that, in the example illustrated in FIG. 3B, the slope part 36 is formed from substantially the center portion of the first face 31 interposed between the conductors 2 facing each other. Alternatively, the slope part 36 may be formed on the outer circumferential side from the portion of the first face 31 interposed between the conductors 2 facing each other (may be formed in a portion close to the clearances 35 in the example illustrated in FIG. 3A). When the slope part 36 is formed in the portion close to the clearances 35, it is possible to easily adjust the surface area of the conductors 2 exposed from the first face 31 in the same manner as in the examples illustrated in FIG. 1 and FIG. 2.

Further, in terms of easier adjustment of the surface area of the conductors 2 exposed from the first face 31, the slope part 36 may be formed to a portion spaced apart from the conductors 2 in the first face 31 interposed between the conductors 2 facing each other. While the slope part 36 is formed in contact with the conductors 2 in the example illustrated in FIG. 3B, it is possible to easily adjust the surface area of the conductors 2 exposed from the first face 31 by forming the slope part 36 to the portion spaced apart from the conductors 2 (in the example illustrated in FIG. 3A, in the direction of the dotted line illustrated by Y-Y′ from the conductors 2).

The protruding parts 34 may be arranged between a plurality of conductors 2. However, since electric pulses are supplied to the conductors 2, it is preferable not to arrange the protruding parts 34 between the conductors 2 exposed in the first face 31 so as to less affect the electric field. Further, it is more preferable to arrange the protruding parts 34 on the outer circumferential side from the conductors 2 exposed in the first face 31. Because the protruding parts 34 are arranged outside the conductors 2, when the electrode 1A is installed to a substrate or the like, a contact portion between the protruding parts 34 and the substrate or the like is increased, and the conductors 2 can be maintained stably in the place where the electrode 1A is installed. Further, when the protruding parts 34 are arranged outside the conductors 2, it is possible to increase the distance between any points of the protruding parts 34. Thus, when the electrode 1A is installed to the substrate or the like, the conductors 2 can be maintained stably in the place where the electrode 1A is installed. FIG. 4 illustrates, not as a limitation, an example in which different protruding parts 34 from those in the example illustrated in FIG. 1 are provided. FIG. 4 illustrates the first face 31 of the member 3 when viewed from front. FIG. 4A illustrates an example in which the protruding parts 34 of the example illustrated in FIG. 1B are arranged with rotation by 90 degrees thereof. FIG. 4B to FIG. 4D illustrate examples in which the number of protruding parts 34 is two, three, and four. Further, as with the example illustrated in FIG. 4D, the protruding parts 34 may be arranged inside the outer circumference of the first face 31. In the example illustrated in FIG. 4, the protruding parts 34 are spaced apart from each other to form the clearances 35. Therefore, when the electrode 1 is put into the buffer, the air around the first face 31 can move along the first face 31 to outside of the member 3 through any of the clearances.

Note that, as described above, in terms of electrical conduction between the conductors 2 facing each other, it is preferable that no air remain between the conductors 2 facing each other. Therefore, as illustrated in FIG. 1B, when, in the outer circumferential portion of the first face 31, a portion interposed between virtual lines IL extending from the conductors 2 facing each other is defined as an outer circumferential virtual region IR, it is preferable that the clearances 35, which are provided by the protruding parts 34 being spaced apart from each other, and the outer circumferential virtual region IR at least partially overlap each other. Because at least a part of the clearances 35 overlaps the outer circumferential virtual region IR, this facilitates the air between the conductors 2 facing each other to escape from the clearances 35 between the protruding parts 34. Note that, although the whole of each clearance 35 is included in the outer circumferential virtual region IR in the example illustrated in FIG. 1B, the clearance 35 may be made larger so as to include the whole outer circumferential virtual region IR as illustrated in FIG. 4B. Further, as illustrated in FIG. 4C, one of the clearances may overlap the outer circumferential virtual region IR.

The electrodes 1A to 1C according to the first embodiment achieve the following advantageous effects.

(1) Since the member 3 of the electrodes 1A to 1C has the protruding parts 34 on the first face 31, when each of the electrodes 1A to 1C is installed to a substrate or the like, the conductors 2 can be maintained stably in the place where each of the electrodes 1A to 1C is installed. Further, the conventional electrode is fabricated by first coating the conductor 81 with the insulator 82 and then holding the conductor 81 coated with the insulator 82 in a holding portion. Thus, as illustrated in FIG. 8, the distance from the holding portion to the tip of the conductor 81 is relatively long. In contrast, in the electrodes 1A to 1C, since the conductors 2 except for the portion exposed from the first face 31 are held by the member 3, the centroid of the electrode in use can be lowered compared to the conventional electrode. Therefore, with the use of the electrodes 1A to 1C, electroporation can be stably performed.

(2) The length by which the protruding part 34 protrudes in the axis L direction from the first face 31 is longer than the length by which the end 21 of the conductor 2 is exposed from the first face 31. Therefore, when each of the electrodes 1A to 1C is used for electroporation on adherent cells, the electroporation can be performed without the conductors 2 being in contact with the adherent cells.

(3) When the slope part 36 is provided to the first face 31 interposed between the conductors 2 facing each other, air moves to the outer circumferential side of the member 3 along the slope part 36 even when the electrode is put into the buffer. Therefore, a likelihood of air remaining between the conductors 2 is reduced.

(4) Since each of the electrodes 1A to 1C can be handled as a separate member from a power supply, the electrodes 1A to 1C can also be used as a disposable electrode.

### Second Embodiment of Electrode

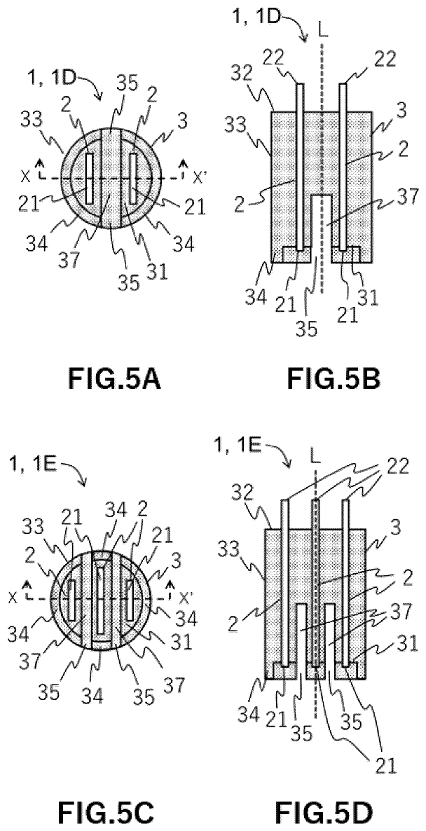

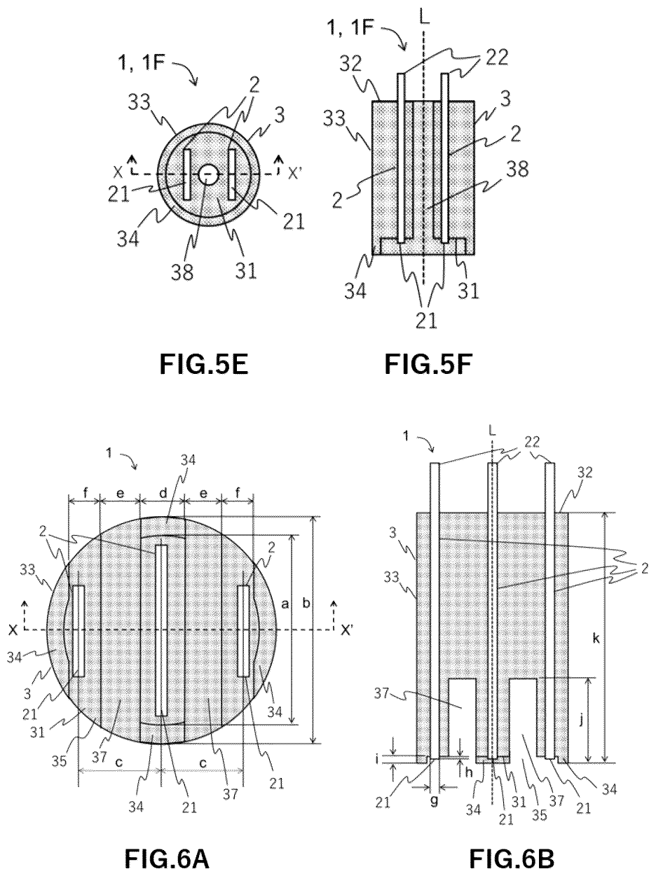

Electrodes 1D to 1F according to the second embodiment will be described with reference to FIG. 5. FIG. 5 represents schematic diagrams illustrating examples of the electrodes 1D to 1F in which the member 3 has recesses 37 or a through hole 38. FIG. 5A is a diagram of the first face 31 of the electrode 1D when viewed from front. FIG. 5B is a sectional view taken along the arrow X-X′ of FIG. 5A. FIG. 5C is a diagram of the first face 31 of the electrode 1E when viewed from front. FIG. 5D is a sectional view taken along the arrow X-X′ of FIG. 5C. FIG. 5E is a diagram of the first face 31 of the electrode 1F when viewed from front. FIG. 5F is a sectional view taken along the arrow X-X′ of FIG. 5E.

The electrodes 1D to 1F according to the second embodiment differ from those of the first embodiment in that the member 3 has the through hole 38 and/or the recesses 37 in the first face 31. Therefore, for the electrodes 1D to 1F according to the second embodiment, features different from those of the first embodiment will be mainly described, and duplicated description for the features that have already been described in the first embodiment will be omitted. Thus, it is apparent that, even when not explicitly described in the second embodiment, the features that have already been described in the first embodiment can be employed.

The recess 37 is provided in the first face 31, is recessed in the axis L direction of the member, and penetrates through in the side face 33 direction of the member 3. Further, the through hole 38 penetrates through the first face 31 and a face other than the first face 31. Because the member 3 has the recesses 37 and/or the through hole 38, the space surrounded by the first face 31 and the protruding parts 34 is connected to outside of the member 3 via the recesses 37 and/or the through hole 38. Therefore, when each of the electrodes 1D to 1F is put into the buffer, air of the space surrounded by the first face 31 and the protruding parts 34 can be more reliably discharged through the recesses 37 and/or the through hole 38.

The number of recesses 37 and through holes 38 and the arrangement of the recesses 37 and the through holes 38 provided in the first face 31 are not particularly limited as long as they can connect the space surrounded by the first face 31 and the protruding parts 34 to outside, and the member 3 may have any one of or both of the recess 37 and the through hole 38.

In the example of the electrodes 1D and 1E illustrated in FIG. 5A to FIG. 5D, the recess 37 recessed in the axis L direction from the first face 31 and penetrating through in the side face 33 direction is provided between the conductors 2. Further, in the example of electrode 1E illustrated in FIG. 5C and FIG. 5D, three conductors 2 are held by the member 3, and two recesses 37 are provided in the member 3. Further, in the example of the electrode 1F illustrated in FIG. 5E and FIG. 5F, the through hole 38 penetrating through the first face 31 and the second face 32 is provided in the member 3. Because the member 3 has the through hole 38 as with the electrode 1F, the member 3 may have a single protruding part 34 not disconnected on the outer circumference of the first face 31. Although not illustrated, the through hole 38 may penetrate from the first face 31 to the side face 33. Note that, although provided between the conductors 2 in the examples of the electrodes 1D to 1F illustrated in FIG. 5, the recess 37 or the through hole 38 may be provided in a place other than the above, for example, provided between each conductor 2 and the outer circumference of the member 3.

The electrodes 1D to 1F according to the second embodiment synergistically achieve the following advantageous effects in addition to the advantageous effects achieved by the electrodes 1A to 1C according to the first embodiment.

(1) The member 3 has the recess(s) 37 and/or the through hole 38, and this enables more reliable discharge of air of the space surrounded by the first face 31 and the protruding parts 34. Therefore, electroporation can be performed without any contact of the conductors 2 exposed in the first face 31 with air.

(2) In the case of the electrodes 1A to 1C according to the first embodiment, the length by which the protruding part 34 protrudes in the axis L direction from the first face 31 is required to be designed in accordance with use. For example, when electroporation is performed on adherent cells, the length by which the protruding part 34 protrudes in the axis L direction from the first face 31 is relatively shorter. Accordingly, the size of each clearance 35, which is formed by the protruding parts 34 being spaced apart from each other, depends on the width of the protruding parts 34. In contrast, in the electrodes 1D to 1F according to the second embodiment, the size of each recess 37 and/or each through hole 38 can be decided regardless of the length by which the protruding part 34 protrudes, and this improves flexibility of design.

### Embodiment of Electrode Kit

An electrode kit has at least any one of the electrodes 1A to 1F and a holder. The electrodes 1A to 1F of the electrode kit have already been described in the above embodiments. Thus, duplicated description will be omitted for the electrodes 1A to 1F.

The holder electrically connects an external power supply to the conductors 2 held by the member 3 of the electrodes 1A to 1F and is used to supply power from the external power supply to the conductors 2. The holder is not particularly limited as long as it can electrically connect the external power supply and the conductors 2 of the member 3 to each other. For example, in the example of the electrode 1A illustrated in FIG. 1, since the conductors 2 protrude from the second face 32 and are exposed, a holder to be fitted to the protruding conductors 2 may be used. Further, in the example of the electrode 1B illustrated in FIG. 2, since the conductors 2 are exposed in the side face 33 of the member 3, a holder contacted with the conductors 2 exposed in the side face 33 of the member 3 to form electrical connection to the conductors 2 may be used. Furthermore, when the conductors 2 electrically connected to the external power supply are not exposed from the member 3 and the member 3 has an insertion hole connected to the conductors 2, the holder can have an insertion part inserted into the insertion hole so as to be electrically connected to the conductors 2.

The electrode kit according to the embodiment is characterized in that the electrode kit includes the electrode 1 according to the above embodiments. Therefore, the same advantageous effects as those of the electrodes 1A to 1F according to the above embodiment are achieved.

Note that the present invention is not limited to the embodiment described above. Within the scope of the present invention, any combination of respective embodiments described above, modification of any component of each embodiment, or omission of any component of each embodiment is possible.

### EXAMPLES

### Example 1

Electroporation was performed on adherent cells by using the electrode 1. The materials, devices, and procedures used will be described below. Further, FIG. 6 illustrates the electrode 1 used. FIG. 6A is a diagram of the first face 31 when viewed from front. FIG. 6B is a sectional view taken along the arrow X-X′ of FIG. 6A. As the electrode 1, an electrode in which three plate-like conductors 2 were held in the member 3 was used. [Material and Device]
African green monkey's kidney cells COS-1 adhered to a 24-well plate
Plasmid DNA incorporating green fluorescent protein (GFP) gene
Opti-MEM I (Invitrogen) (buffer)
Cell culture medium for post-culture (DMEM medium (Sigma)+10% FBS (Thermo) penicillin-streptomycin mixed solution (Nacalai Tesque) diluted 100-fold addition)
Electroporator (CUY21EDIT II (BEX CO., LTD.)
Electrode 1 (Each size of a to k illustrated in FIG. 6 is listed in Table 1)
Fluorescent microscope (Nikon DIAPHOTO300, Nikon HB-10103AF, ultra-high pressure mercury lamp power supply device)

TABLE 1

| | size (mm) |
|---|---|
| a | 12.5 |
| b | 14.5 |
| c | 5.5 |
| d | 2.5 |
| e | 3 |
| f | 1.75 |
| g | 2 |
| h | 0.1 |
| i | 0.6 |
| j | 8 |
| k | 22 |

### Procedure

1. Plasmid DNA was added to the buffer to give plasmid DNA concentration of 5 μg/μL.

2. The wells to which cells were adhered were washed by the buffer.

3. The buffer of 250 μL including the plasmid DNA prepared in 1. was added to the wells.

4. The electrode 1 was arranged in the well so that the cells were not detached therefrom.

5. Electroporation was performed. In the electroporation, the electrode 1 was connected to the electroporator such that the center conductor 2 was positive, and two conductors on both sides were negative, and electric pulses were supplied to the cells in a damped wave (Decay (V)) mode. The poration pulse (Pp) and the driving pulse (Pd) in the damped wave mode were set as follows.
Pd setting value
   Voltage: 250V
   Pulse duration (Pon): 10 ms
   Pulse interval (Poff): 50 ms
Pd setting value
   Voltage: 30V
   Pulse duration (Pon): 50 ms
   Pulse interval (Poff): 50 ms
   Number of pulses: 5
   Pattern: +/−
   Capacitance: 940 μF

6. The electrode 1 was taken out of the well, and the well plate was incubated for 10 minutes in a CO₂ incubator for recovering the cells.

7. The buffer was removed from the well, and a pre-incubated cell culture medium for post-culture was gently added thereto.

8. The well plate was moved to the CO₂ incubator, and post-culture was started.

9. After 48 hours of the post-culture, the well plate was taken out of the CO₂ incubator, and the cells were observed by using the fluorescent microscope.

### Comparative Example 1

Comparative example 1 is the same as Example 1 except that the electrode used in electroporation was the conventional electrode with legs (LF513-5, BEX CO., LTD.).

With respect to the electrode 1 used in Example 1, when the electrode 1 was arranged in the well, the conductors 2 was maintained at a predetermined position stably due to the protruding parts 34 of the member 3. Further, since the tips of the protruding parts 34 protrude more than the ends 21 of the conductors 2 exposed in the first face 31 of the member 3 of the electrode 1, the conductors 2 did not come into contact with cells. Therefore, in Example 1, with the use of the electrode 1, the conductors 2 was maintained at a predetermined position, and the electroporation on adherent cells was stably performed. In contrast, in Comparative example 1, since the conventional electrode with legs was used, it was difficult to stably maintain the electrode with legs at a predetermined position.

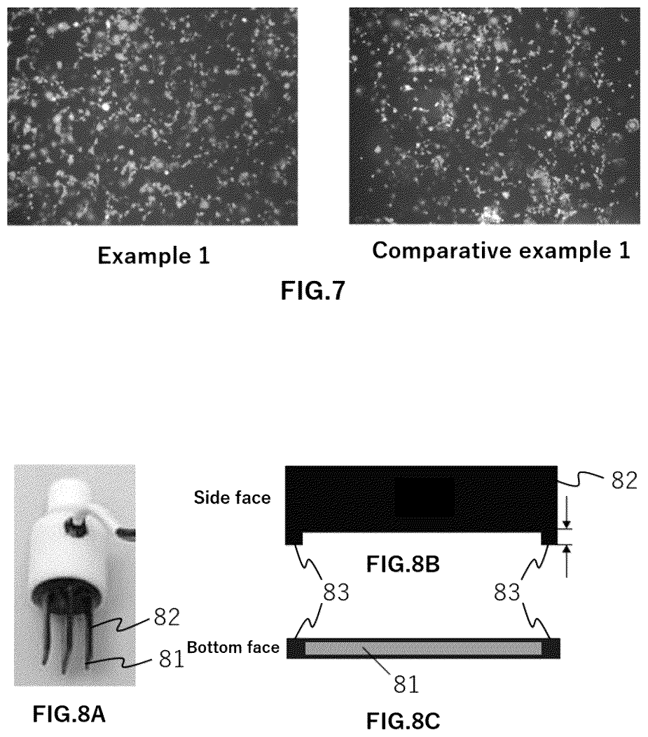

After the electroporation was performed, the post-cultured adherent cells were observed by using the fluorescent microscope. FIG. 7 illustrates the result. It was indicated in FIG. 7 that, in both Example 1 and Comparative example 1, plasmid DNA was introduced to the adherent cells by electroporation, and GFP was expressed. It was therefore shown that the electrode 1 used in Example 1 can perform electroporation at efficiency to the same degree as the conventional electrode with legs. Thus, it was shown that the electrode 1 can maintain the position the conductors more stably than the conventional electrode with legs and can introduce genes to adherent cells to the same degree as the conventional electrode with legs, and therefore, the electrode 1 can perform electroporation in a simple manner.

### INDUSTRIAL APPLICABILITY

The use of the electrode and the electrode kit disclosed in the present application makes it easier to arrange the electrode at a predetermined position when performing electroporation on cells. Therefore, the electrode and the electrode kit are useful for business entities that handle an electrode used for performing electroporation on cells.

### LIST OF REFERENCES

1, 1A to 1F
electrode
2 conductor
21, 22 end
3 member
31 first face
32 second face
33 side face
34 protruding part
35 clearance
36 slope part
36a slope face
37 recess
38 through hole
81 conductor
82 insulator
83 leg
IL virtual line
IR outer circumferential virtual region
L axis

1. An electrode used for performing electroporation, the electrode comprising:
at least two or more conductors; and
a member configured to hold at least a portion of each conductor inside and protrude at least one end of each of the conductors to outside,
wherein the member has
a first face where the one end of each of the conductors is protruded, and
at least one or more protruding parts provided to the first face, and
a recess provided in the first face, and
wherein the recess is recessed in an axis direction of the member from the first face and penetrates through in a side face direction of the member,
wherein the length by which the one or more protruding parts protrude in an axis direction from the first face is longer than the length by which the one end of each of the conductors is protruded from the first face.

2. The electrode according to claim 1, wherein the one or more protruding parts comprise two or more protruding parts, and the two or more protruding parts are spaced apart from each other.

3. The electrode according to claim 2, wherein when, in an outer circumferential portion of the first face, a portion interposed between virtual lines extending from the conductors facing each other is defined as an outer circumferential virtual region,
a clearance, which is provided by the protruding parts being spaced apart from each other, and the outer circumferential virtual region at least partially overlap each other.

4. The electrode according to claim 1, wherein a slope part is provided to the first face.

5. The electrode according to claim 2, wherein a slope part is provided to the first face.

6. The electrode according to claim 3, wherein a slope part is provided to the first face.

7. The electrode according to claim 1, wherein the one or more protruding parts are arranged on the outer circumferential side from each of the conductors protruded in the first face.

8. The electrode according to claim 1,
wherein the member has a through hole,
wherein the through hole penetrates between the first face and a face other than the first face.

9. The electrode according to claim 7,
wherein the member has a through hole,
wherein the through hole penetrates between the first face and a face other than the first face.

10. An electrode kit comprising:
the electrode according to claim 1; and
a holder connected to a power supply,
wherein the other end of each of the conductors is electrically connected to the holder, and power is supplied to each of the conductors from the power supply.

11. An electrode kit comprising:
the electrode according to claim 2; and
a holder connected to a power supply,
wherein the other end of each of the conductors is electrically connected to the holder, and power is supplied to each of the conductors from the power supply.

12. An electrode kit comprising:
the electrode according to claim 3; and
a holder connected to a power supply,
wherein the other end of each of the conductors is electrically connected to the holder, and power is supplied to each of the conductors from the power supply.

13. An electrode kit comprising:
the electrode according to claim 4; and
a holder connected to a power supply,
wherein the other end of each of the conductors is electrically connected to the holder, and power is supplied to each of the conductors from the power supply.

14. The electrode according to claim 2, wherein the one or more protruding parts are arranged on the outer circumferential side from each of the conductors protruded in the first face.

15. The electrode according to claim 3, wherein the one or more protruding parts are arranged on the outer circumferential side from each of the conductors protruded in the first face.

16. The electrode according to claim 4, wherein the one or more protruding parts are arranged on the outer circumferential side from each of the conductors protruded in the first face.

17. The electrode according to claim 5, wherein the one or more protruding parts are arranged on the outer circumferential side from each of the conductors protruded in the first face.

18. The electrode according to claim 6, wherein the one or more protruding parts are arranged on the outer circumferential side from each of the conductors protruded in the first face.

19. The electrode according to claim 2,
wherein the member has a through hole,
wherein the through hole penetrates between the first face and a face other than the first face.

20. The electrode according to claim 3,
wherein the member has a through hole,
wherein the through hole penetrates between the first face and a face other than the first face.
